(19) United States
(12) Patent Application Publication
(10) Pub. No.: US 2026/0041354 A1
(43) Pub. Date: Feb. 12, 2026

(54) STRETCHABLE TEXTILE SENSOR WEARABLE DEVICE FOR TRACKING ONE OR MORE BODY METRICS

(71) Applicant: TEXAVIE TECHNOLOGIES INC., Vancouver (CA)

(72) Inventors: Peyman SERVATI, Vancouver (CA); Arvin TASHAKORI, Vancouver (CA); Amir SERVATI, Vancouver (CA); Zenan JIANG, Vancouver (CA); Saeid SOLTANIAN, Vancouver (CA); Harishkumar NARAYANA, Vancouver (CA); Amir POURNAJIB, Vancouver (CA); Caroline NAKAYAMA, Vancouver (CA); Menghan MA, Vancouver (CA); Xueyi LI, Vancouver (CA)

(21) Appl. No.: 19/126,896

(22) PCT Filed: Nov. 3, 2023
(86) PCT No.: PCT/CA2023/051473

§ 371 Date: May 2, 2025

Related U.S. Application Data
(60) Provisional application No. 63/422,867, filed on Nov. 4, 2022.

(51) Int. Cl. A61B 5/27; A61B 5/00; A61B 5/0205; A61B 5/11; A61B 5/318; A61B 5/389; A61B 5/398

(52) U.S. Cl. CPC A61B 5/27; A61B 5/0022; A61B 5/0205; A61B 5/112; A61B 5/1127; A61B 5/1128; A61B 5/318; A61B 5/389; A61B 5/398; A61B 5/6806; A61B 5/742; A61B 2560/0214; A61B 2562/0219

(57) ABSTRACT

This invention relates to smart textile wearable device with embedded yarn sensors and a computing device comprising a processor and a computer readable medium having encoded thereon a machine learning program that gathers sensor data from the wearable device and generates health parameter output data or real time or long-term feedback to a user for interaction, health and exercise purposes.

### FIELD

This invention relates to stretchable textile smart sensor apparel and other wearable devices for tracking one or more body metrics.

### BACKGROUND

Accurate and dynamic tracking of hand and body movements and strength is critical in many applications including human-computer interaction (HCl), electronic gaming, animation and movie making, metaverse, augmented reality (AR), virtual reality (VR), mixed reality (MR), robotics, robot and human interactions, sports training, sports injury assessment and therapy, tele-health, tele-rehabilitation, prosthetics, chronic disease management, and tele-surgical applications. Accurate and dynamic tracking of hand and fingers is used for user interaction in metaverse, AR/VR/MR, animation, controlling robots and performing surgery or tele-surgical procedures. Accurate tracking also can be used in sports training and simulation applications where performance of the hands in handling a ball, a bat, a stick or other sports equipment is important to the outcome. In addition, accurate and dynamic tracking of hands are important for dexterous hand movement assessment during diagnosis and long-term exercise therapy for patients who have suffered from any hand impairments after stroke, traumatic brain injury (TBI), rheumatoid arthritis (RA), Parkinson's, carpal tunnel or any other neuromuscular, neurodegenerative, or joint injuries. Accurate and dynamic tracking of hand, fingers and their muscles enables experts including physiotherapist or clinical specialist to diagnose conditions and triage remotely, check the compliance of patients in performing their home exercise plan, modify their exercise plans or prescriptions based on personalized assessment and achieve better outcomes. In addition to movement, other parameters such as grasp forces, object manipulation capabilities, temperature, sweat, heart rate and blood pressure can be important to assess cognitive load and physiological conditions during a specific exercise or movement or task that is important for tele-health and tele-rehabilitation applications.

Similar accurate and dynamic tracking is required for body movements during exercises with applications in sports, fitness and home exercise, assessment and diagnosis of joint health and muscle conditions for all body joints, fitness level, cardio rehabilitation, assessment of balance and mobility, interaction with prosthetics, assessment of orthopedic impants, assessment of chronic disease conditions, and progression of pregnancy. For example, the knee joint is central to lower body function during walking, running or performing work duties or daily activities. Accurate and dynamic tracking of knee joint or other bodily joint movements and assessment of muscles and their strength is important for patients that have suffered from a sport injury, Anterior Cruciate Ligament (ACL) injury, stroke, RA, Parkinson's or any other neuromuscular or neurodegenerative or genetic conditions that affect their mobility and function. Dynamic and accurate tracking enables experts such as physiotherapists, clinical specialist or fitness coaches assess, diagnose or triage conditions remotely, check for the compliance of patients in following home exercise plans and modify their plans, prescriptions, exercises and targets based on personalized assessment and help patients be engaged in their therapy progression. In addition, other health parameters such as body temperature, sweat, heart rate, heart rate variability, cardiac output, respiratory rate and blood pressure can provide information that extends joint assessment to an overall assessment of health conditions. Providing accurate movement and muscle information in addition to physiological parameters help in accurate assessment of exercise and therapy with applications in accurate remote cardio rehabilitation and joint and overall body assessment. These technologies can be used for people in different age groups including elderly and seniors, adults, teens, kids and babies, and women and men, pregnant women, and other groups to provide feedback.

These technologies are critical for providing better access to care for both hands and body to people living in suburban areas, rural areas, and remote areas where access to an expert is not easy to schedule and there can be long and costly commute. Such tele-health and tele-rehab technologies can enable scheduling data-rich virtual supervised sessions, offline unsupervised sessions and machine-learning autonomous remote assessment and therapy. In addition to remote and inaccessible areas these technologies can be used for providing assessment and therapy for travelers on plane, ships or other people in transport or astronauts and space tourists. These technologies can be specially designed for being lightweight and durable for operation during space mission as part of space suits and individual healthcare kits.

Current accurate movement tracking devices rely on expensive fixed motion capture cameras and marker systems for tracking movement of hands, fingers, and other body parts. These systems are known to have a long set up time, limited field of view (FoV) bound by the fixed positions of the cameras and are prone to occlusion by objects or body parts and environmental optical noise and light intensity. Computer vision (CV) systems are also developed as lower cost consumer products that can be mobile and estimate body and hand movements but rely on several cameras placed on headsets or in a room and connected to extensive computing processors for running CV algorithms, provide limited accuracy, and still have issues related to limited FoV, occlusion, and optical noise. These cameras include optical cameras, infrared cameras, infrared laser cameras, depth cameras and other cameras used to create video output of the user from different angles. Wearable devices based on inertial measurement units (IMUs), global positions systems (GPS), electromyography (EMG) or pressure and stretch sensor gloves or other apparel have also been shown to provide limited accuracy and have been demonstrated only for limited gesture recognition and not for accurate dynamic tracking and interaction with objects. In addition, these wearable devices are not particularly stretchable, breathable, washable, or sanitizable and have significant issues with the variation in fit for the same or different users, resulting in impracticality for real home, clinical or outdoor use.

In addition, most CV, camera, and wearable systems have not been shown to assess individual muscle strength, form, symmetry, and movement quality dynamically and accurately during exercises. Providing muscle strength, volume and symmetry data and other health parameters are important for overall assessment of exercise, personalization of therapy progression, and developing effective remote therapy and health monitoring applications as well as applications in gaming, metaverse, object interaction and assessment of forces in movements.

In addition, most known wearable devices are bulky, not durable, not washable and cannot be easily worn or set up by an average consumer or user. These are critical aspects that are necessary for practical and commercial use of such devices at home, outdoors or in different clinical conditions. In addition, most wearable devices are not designed to perform accurately considering the different fit for different users and body forms or the same user in different sessions. This has made these solutions impractical and unusable.

Some exemplary patents that describe the current state of the art for wearable systems include:

U.S. Pat. No. 11,137,298 by Pan et al. presents a fabric-based pressure sensing gloves; however, this system is not washable and cannot perform object interaction and dynamic tracking of the hand movements.

US Patent 2021/0373663 A1 by Matusik et al. presents a knitted fabric-based pressure sensing device in form of a glove; however, the sensors are not insulated, the device is not washable and prone to noise if person's hand is sweating. In addition, it cannot perform object interaction and dynamic tracking and orientation of hand and joints with the accuracy of the work disclosed herein and ML does not include any robustness or mutli-level ML for core motion and output.

### BRIEF SUMMARY OF THE INVENTION

Aspects of the invention relate to stretchable, washable, and breathable smart textile wearable devices and smart apparel with embedded lightweight yarn sensors that can be easily worn by users, patients or consumers and help in accurate and dynamic tracking of body and hand movements, muscle strength and volume evaluations and measurement of other health parameters. The system can be wirelessly (e.g., WiFi, Bluetooth, 5G, 6G, LTE, or other communication protocols) connected to a data gateway (e.g., phone, smartwatch, tablet, computer, gaming console, smart WiFi, smart headphones, smart speakers or smart TV) and provide real-time feedback and interactive features or be used to provide long term feedback or used for diagnosis and assessment of conditions for over a period of time or during long-term therapy. Different sensor modalities in form of stretch and force yarn sensors, bio-signal yarn sensors, temperature and sweat yarn sensors, inertial measurement units (IMUs) or photoexcitation and detection sensors (e.g., photoplethysmography (PPG) or near-infra-red spectroscopy (NIRS) sensors) are embedded into the smart apparel and interconnected with stretchy interconnects. The data from smart apparel can be transmitted wirelessly and processed by a core machine learning (ML) program that generates accurate and dynamic joint angles, individual muscle strengths, volume and form, movement quality output (e.g., movement strength, power, speed, torque, smoothness, consistency, tiredness, exertion level, effort level, intensity, and symmetry) as well as other health parameters (e.g., heart rate, hear rate variability, blood oxygenation level, respiratory rate, blood pressure). The accurate and dynamic output are not limited by FoV of cameras and can be collected anywhere indoors, outdoors or with poor or limited lighting and most importantly are not prone to occlusion by objects and body parts. In addition, computer vision and some camera data can be used to complement the wearable devices for providing greater accuracy, positioning of different users, calibration of wearables, object interaction, force and strength mapping, or provide reliable continuity even when the subject moves out of the FoV of camera or occluded or in outdoor environments.

According to one aspect of the invention there is provided a wearable device for tracking one or more body metrics, comprising: a textile layer configured to be worn on a body part of a user; at least one body metric sensor including a yarn sensor attached to a location on the textile layer suitable to track a target body metric when the textile layer is worn by the user, and an integrated circuit with at least one stretchable interconnect communicatively coupled to the at least one body metric sensor. The yarn sensor can comprise one or more of a stretch sensing yarn sensor, a force sensing yarn sensor, a temperature sensing yarn sensor, a moisture sensing yarn sensor, a surface electromyography (sEMG) bio-signal yarn sensor or an electrocardiograma-signal yarn sensor. The integrated circuit can comprise a wireless transmitter. The textile layer can be configured to be one or more of a glove, wrist band, knee sleeve, shirt, tights, socks, shoe insole, or torso band.

The stretch sensing yarn sensor can comprise an electrically conductive helical sensor yarn comprising a central stretchable core yarn, a helical nanofiber winding around the core yarn, a metal coating on the helical nanofiber winding and core yarn, and an outer sheath encapsulating the core yarn, helical nanofiber winding, and metal coating. The core yarn can have a composition comprising polyurethane, the helical nanofiber winding has a composition comprising polyacrylonitrile (PAN), the metal coating has a composition comprising gold or an alloy thereof, and the sheath has a composition comprising an elastomer. The stretch sensing yarn sensor can alternatively comprise a composite yarn having a composition comprising conductive nanoparticles and an elastomer.

The force sensing yarn sensor can be a capacitive force sensor comprising a pair of electrodes separated by an elastomer or by an elastomer perimeter with an empty separation between the electrodes. Alternatively, the force sensing yarn sensor can be a triboelectric force sensor. Multiple force sensing yarn sensors can be grouped together in an array that is attached to a location on the textile layer in the vicinity of a body part extremity when worn by the user.

The temperature sensing yarn sensor can comprise electrically resistive yarns that changes resistance as a function of temperature change, or triboelectric yarns connected to an elastomer that changes dimension as a function of temperature change. The moisture sensing yarn sensor can have a composition that changes resistance as a function of water absorption.

The wearable device can further comprise at least one inertial measurement unit (IMU) sensor attached to the textile layer and operable to output spatial data of the body part wearing the wearable device, a photoplethysmography (PPG) or near-infra-red sensor (NIRS) bio-signal detector attached to the textile layer in the vicinity of a blood vessel when worn by the user, and/or a bio-signal sensing yarn sensor comprising Ag or AgCl coated yarns that is operable to measure one or more of electrocardiogra signals, electromyography (EMG) signals, electroencephalography (EEG) signals, electrooculography (EOG) signals, or electrodermal activity (EDA) signals.

According to another aspect of the invention, there is provided a system for tracking, processing and displaying body metrics, comprising the aforementioned wearable device, and a computing device comprising a processor and a non-transitory computer readable medium having stored thereon a trained core machine learning (ML) program executable by the processor to receive raw body metric data from the at least one body metric sensor, correlate the raw body metric data with a corresponding health parameter output stored in a first training dataset, and display the corresponding health parameter output. The computing device can further comprise an output ML program communicative with the core ML program and executable by the processor to receive health parameter output from the core ML program, correlate the health parameter output with a corresponding gesture in a second training dataset, and display the corresponding gesture in a gesture-based application. The first training dataset can be produced by simultaneously collecting body metric data tracked by an external device and by the at least one body metric sensor on the wearable device, while the wearable device is performing specified movements with known health parameter outputs. The external device can comprise one or more motion capture cameras and the wearable device comprises markers; during training of the core ML program, the one or more motion camera cameras track the markers when the wearable device is performing the specified movements.

The textile layer can be configured as a knee sleeve, in which case the one or more yarn sensors includes a stretch sensing yarn sensor, a force sensing yarn sensor, and an IMU sensor, and the health parameter output includes one or more of knee joint angle, range of motion of knee or other lower body joints, valus/vargus movements, speed and torque of movements, timing of movements, repetition of movements, consistency in timing, or individual muscle volume, force, strength, activity, and symmetry between the two legs.

The textile layer can be configured as a shoe insert, in which case the one or more yarn sensors include a stretch sensing yarn sensor, a force sensing yarn sensor, and an IMU sensor, and the health parameter output includes movement parameters including steps, pace, gait, landing pressure and symmetry and consistency between the two legs.

The textile layer can be configured as a torso band in which case the one or more yarn sensors include a stretch sensing yarn sensor, force sensing yarn sensor, temperature sensing yarn sensor, moisture sensing yarn sensor, sEMG bio-signal yarn sensor and ECG bio-signal yarn sensor, and the health parameter output includes spine angle, slouching, movement and strength of core muscles, shape and form of waist and belly, movements of fetus during pregnancy, breathing rate, heart rate, heart rate variability, blood pressure and ECG.

The textile layer can be configured as a glove, in which case the one or more yarn sensors include a stretch sensing yarn sensor, force sensing yarn sensor, and the health parameter output includes wrist angle, finger joint angles, finger pinching force, gesture of the hand, pose of the two hands, grasp force, and object touch force.

The textile layer can be configured as a wrist band comprising a photoplethysmography (PPG) or near-infra-red sensor (NIRS) bio-signal detector and a stretch sensing yarn sensor, force sensing yarn sensor, sEMG bio-signal yarn sensor and ECG bio-signal yarn sensor, and the health parameter output includes hands or fingers movement or force, gestures of the hands, heart rate, blood pressure, and ECG.

The wearable device can comprise at least one IMU sensor and a stretch sensing yarn sensor or a force sensing yarn sensor, in which case the core ML program is further executable by the processor to calibrate the IMU using sensor data collected from one or more of the stretch sensing yarn sensor, force sensing yarn sensor or external camera, by comparing the collected sensor data to IMU signals to determine IMU signal drift or drift or noise from any other sensor modalities.

### BRIEF DESCRIPTION OF THE SEVERAL VIEWS OF THE DRAWINGS

FIG. 1(a) is a schematic of a smart textile wearable device according to one embodiment, namely a smart textile glove with embedded sensor yarns for sensing body metrics including movement and force and having wireless connection means to a data gateway or electronic feedback system. FIG. 1(b) is a schematic of an embodiment of a smart phone displaying processed output data received from the smart textile glove.

FIG. 2(a) is a perspective cut-away view of a stretch sensing yarn sensor of the smart textile glove, according to an embodiment. FIG. 2(b) are graphs illustrating the dynamic strain range of the stretch sensing yarn sensor. FIG. 2(c) are graphs illustrating the time response range of the stretch sensing yarn sensor. FIG. 2(d) are graphs illustrating the performance of the stretch sensing yarn sensor over time and in water and air. FIG. 2(e) is a time-resistance graph of a cellulosic type moisture and sweat yarn sensor according to an embodiment compared to a conventional humidity sensor.

FIG. 3 are photomicrographs depicting an example implementation of the stretch sensing yarn sensors.

FIG. 4 is a graph of the washability and durability test performance of the example implementation of the stretch sensing yarn sensor.

FIG. 5(a) is a schematic block diagram of components of a core machine learning (ML) program. FIG. 5(b) is a graph illustrating the inter-subject and intra-subject accuracy of the tracking of body metrics after training the ML program, as a function of root mean square error (RMSE). FIG. 5(c) is a schematic block diagram of a data-augmentation process for a self-supervised pre-training to increase robustness of the core ML program for predicting results in the presence of external noises such as sensor malfunction/masking, scaling and noise. FIG. 5(d) is a graph illustrating the improved output accuracy of the core ML program augmented by the self-supervised pre-training procedure.

FIG. 6(a) is a schematic block diagram of a core ML program and output ML program for generating output for specific applications without the need for retraining the core ML program. FIG. 6(b) are a photograph, table and graph illustrating an example implementation using the gloves for typing detection on a mock paper keyboard (photograph), the color-coded accuracy of the type actions (table), and click and touch force sensing response performance (graph). FIG. 6(c) are a photograph and graphs of an example implementation of in-air drawing based on wrist angle and pinching of different fingers with thumb for selecting different colors. FIG. 6(d) are photographs and a table of an example implementation showing complex gesture recognition (photographs) and its confusion matrix (table) based on training of output ML model. FIG. 6(e) are photographs and a table of an example implementation showing different grasped objects (photographs) and its confusion matrix (table) based on the training of the output ML model.

FIG. 7 is a block diagram of a data collection procedure to produce a training dataset for training the core ML program.

FIG. 8 illustrates steps for training the core ML program using the collected training dataset.

FIG. 9 a block diagram of a real-time inference pipeline for the trained core ML program and an output ML program, utilizing data received from the smart textile wearable device.

FIG. 10 is a schematic view of smart textile wearable devices according to other embodiments, including smart textile knee sleeves, smart textile shoe insoles and smart textile torso bands.

### DETAILED DESCRIPTION OF EMBODIMENTS OF THE INVENTION

Embodiments described herein relate to a wearable device comprising one or more stretchable textile sensors (“smart textile wearable device”) for tracking one or more body metrics such as movement, muscle strength, force, temperature, sweat, heart rate, blood pressure, electrocardiogramals, electromyography (EMG) signals, electroencephalography (EEG) signals, and electrodermal activity (EDA) signals. Embodiments also include a system comprising the smart textile wearable device and a processor with a non-transitory computer readable medium comprising a trained core machine learning (“ML”) program (“ML processor”) that is executable to process the body metrics tracked by the smart textile wearable device to produce health parameter outputs such as joint angles, muscle properties, and movement quality. The computer readable medium can also include a trained output ML program that is executable to process the health parameter outputs from the core ML program for use in certain gesture-based applications. The ML processor can be located remotely from or optionally integrated in the smart textile wearable device.

An embodiment of this invention is a multimodal smart glove that can be easily and comfortably worn by the user and enable accurate capture of dynamic movements including finger and wrist joints, arm and compensational movements, forces exerted by the fingers and palm during grasp and interaction with different objects, and estimation of muscle strength and movement quality. This smart glove solution is important for electronic gaming, sport training, assessment, and simulation, metaverse, AR, VR, and MR applications as well as robotics, robotic control, and remote surgery. In addition, the smart glove is important for a variety of application including assessment of hand strength for post-stroke, TBI, neuromuscular diseases, neurodegenerative diseases, arthritis, carpal tunnel, and Parkinson's diseases. This enables remote virtual care and assessment for these patients and diagnosis and modification of therapy depending on personalized health conditions. The invention can provide feedback about the strength of the patients hand and fingers based on clinical evaluations of strength, symmetry of hand function, strength of fingers, and evaluate the progress of the patient during long term therapy anywhere at home, outdoors remotely. By using inverse kinematics and machine learning, the strength of movements can be associated to the strength of individual muscles or injuries or conditions associated to individual muscles.

This embodiment enables tracking of hands, fingers or body parts even during interaction with different objects and provide realistic digital representation even in these cases where camera systems are incapable of providing output. As embodiments of this invention, the technology enables advanced applications including typing on mock keyboard or in-air drawing or object recognition in addition to accurate dynamic tracking of hands and fingers. An output ML program is used to tune the output of the core ML model for specific applications including keyboard, handling of ball, therapy and provide objective real-time or long-term feedback. In addition, the multimodal sensor yarns can capture changes in sweat and temperature that can be indication of mental and cognitive loads as well as biometric signs for joint health and condition in arthritis or other disease conditions. The multimodal sensors can be used to detect electrocardiogram (ECG), blood pressure, electromyography (EMG) or other health parameters that can augment the movement data for better personalized assessment of patient and providing diagnosis and triage even remotely. Another embodiment of this invention is a multimodal smart wristband or armband that can be easily and comfortably worn by the user and enable accurate capture of dynamic movements including finger and wrist joints, arm and compensational movements, as well as heart rate, ECG and blood pressure, from the stretch sensor yarns, EMG biosignal electrode yarns, ECG biosignal yarns, and PPG sensors embedded in the same wristband or armband.

Other embodiments of the invention can be in form of smart knee sleeves, shoe insoles, torso or chest band or bands, arm sleeves, shoulder sleeves, neck sleeves, wrist band, hat, headband, or other complete forms such as smart shirt, pants, shorts, socks, or suits. Such multimodal devices can gather dynamic information about joint movements including accurate flexion/extension of joints, odd or irregular movement of joints, individual muscle strength and volume, movement strength, symmetry of movements, smoothness of movement, intensity of movement and exercise, power, and calories. An example embodiment, the smart knee sleeves can monitor the flexion/extension angles and Range Of Motion (ROM) of the two knee joints during different movements and exercises dynamically and provide feedback if the exercise timing and targets are met. The angle tracking can be accurate and in different orientations extracted from IMUs or complemented with computer vision using camera as well as stretch and force sensors and can be for normal flexion/extension or abnormal conditions such as hyper extension, and valus/vargus rotation angles. Angles for hip and ankle joints can also be extracted from knee sleeves using core ML program. The smart knee sleeves can be used to extract the information about dynamic volume changes, form, power and strength of individual muscles such as quadriceps, hamstrings, calves, and shin or other muscle groups. The smart knee sleeves can provide information about symmetry in movement, speed, muscle strength and movement quality between two legs for more effective exercise assessment and therapy feedback. Personalized suggestions and feedback on the type of movements can be made using machine learning and Al to compare the user to trained model from large population of people including athletes and experts. Another example embodiment, the smart shoe insoles can be used to map the foot pressure points, gait and pace parameters during walking, running and other movements anywhere at home, outdoors or in clinic. Another example embodiment is the smart chest or torso bands that can be used to detect the spine and back movements during different exercises, ECG, heart rate, respiratory rate, or other health conditions including fetal movements for pregnant mothers. Several forms can be used at the same time or other form factors can provide similar assessment and feedback depending which body parts.

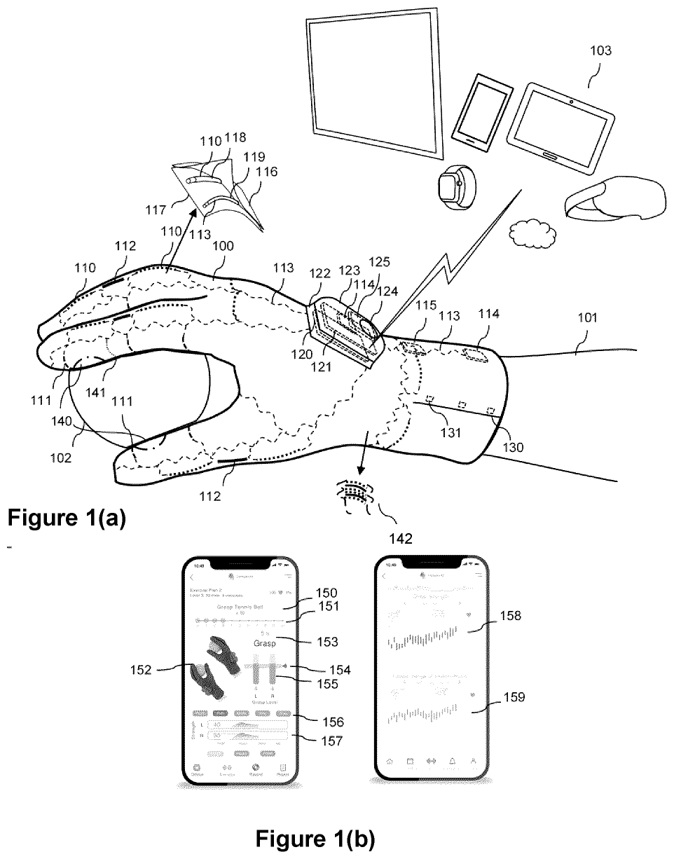

An embodiment of the smart textile wearable device is illustrated in FIG. 1(a), which is a smart textile glove 100 worn on the hand of a user 101 who is holding an object 102 and wirelessly connected to a data gateway and feedback device 103. The smart textile glove 100 comprises stretchable inner and outer textile layers 116, 117 sandwiching and embedded with yarn sensors with different sensing modalities, including: stretch sensing yarn sensors 110, force sensing yarn sensors 111 and bio-signal sensing yarn sensors 112 for sensing temperature, sweat and other biological metrics. The yarn sensors 110, 111 112 are located in the glove 110 where the desired body metrics can be measured when the glove is worn, and for example can be located in the proximity of finger, wrist joints, palm of the hand, tips of the fingers or around the wrist when the glove is worn. The yarn sensors 110, 111, 112 are communicatively connected by vine-like stretchy interconnects 113 that electrically connect to each yarn sensor individually and are embedded in the textile layers 116, 117 without making limitations in stretchability, comfort, breathability, washability and fit for the user. The stretchy interconnects 113 communicatively connect the yarn sensors 110, 111, 112 to an embedded integrated circuit 120 in the smart textile glove 100 that serves in processing and/or wirelessly communicating with the data gateway and feedback device 103.

Inertial measurement unit (IMU) sensors 114 can be embedded in the textile layers 116, 117 and connected to an embedded integrated circuit 120 using the stretchy interconnects 113. The IMU sensors 114 can include commercially available three axes accelerometers, three axes gyroscopes and three axes magnetometers that provide raw spatial data for accelerations and orientation in space. As will be discussed further below, this spatial data is processed to calculate the angles and positioning of the IMU sensors 114 and create quaternions for 3D movements of the points in space, which then can be used to extract accurate angles of body parts that are wearing the smart textile wearable device, such as flexion and extension angles and other angles including valgus/varus rotation, medial/lateral rotations and hyper-extension. For example, two IMUs 114 embedded in the smart textile glove 100 can be used to accurately generate angles of rotation of wrist including pronation, supination, flexion and extension. Camera and computer vision can be used for improving accuracy, decreasing drift and calibration of IMUs during the use of system. Other modalities such as GPS and other sensors can be added to reduce drift and improve accuracy and continuity of data for the IMUs.

In some embodiments, a photoplethysmography (PPG) or near-infra-red sensor (NIRS) bio-signal detector 115 is included in the smart textile glove 100 and is connected to the integrated circuit 120 for evaluation of blood flow, blood oxygenation or other parameters used for evaluation of heart rate, blood pressure or health of the user. Light-emitting diodes of the bio-signal detector 115 emit light to blood vessels in the hand and the reflections are detected by photodetectors of the bio-signal detector 115. Commercially available photodetector diodes and light-emitting diodes can be used in the bio-signal detector 115 to detect and generate different spectrum of light including different spectrum of visible light and infrared. Infrared or red lights are known to have more penetration and provide information about oxygenation and hemoglobin content in the blood or tissue. In some embodiments, small dimension photodetectors or light emitting diodes (including micro-photodiode and micro-LEDs, organic LEDs, or quantum dot LEDs) are integrated on a small flexible substrate or directly attached to stretchable wire electrodes and placed on a desired location of the textile layers 116, 117 facing the body part of interest when the wearable device 100 is worn. The photodetector and light emitting diodes can also be coupled with optical fibers or lens coatings (not shown) to better distribute or focus light in the desired directions to interact with the body part. This can be used for detection of oxygen saturation (SpO2) and oxygenation of the blood, blood pressure, heart rate, and oxygenation of tissue and muscles.

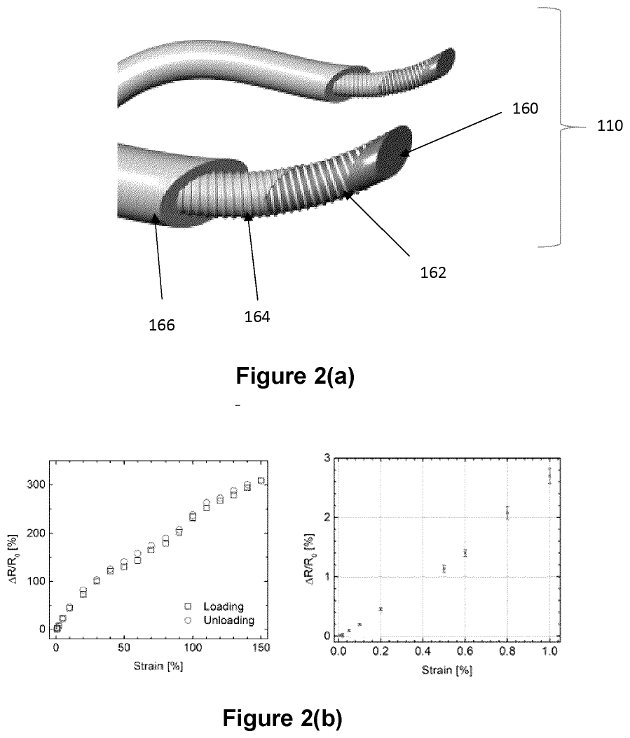

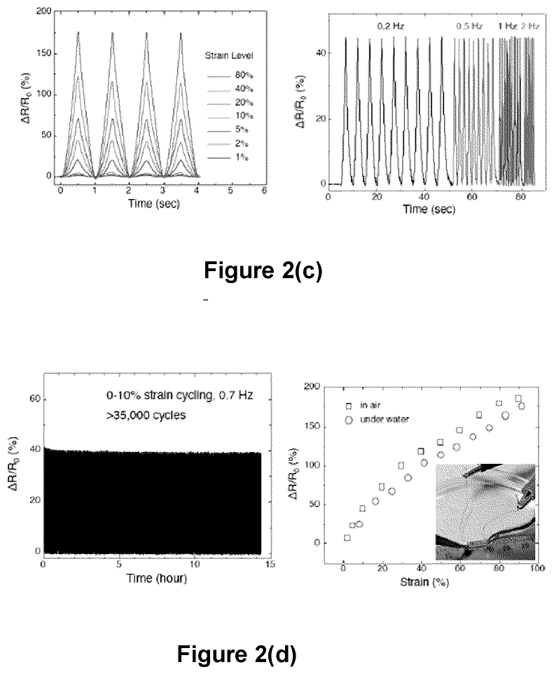

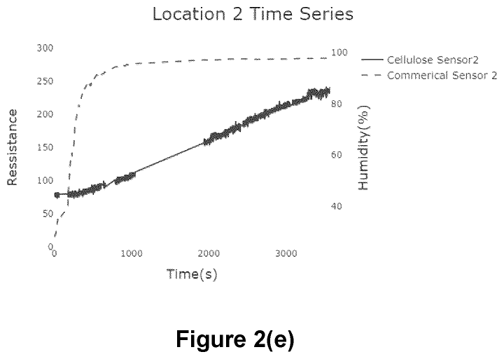

Referring now to FIG. 2(a), the stretch sensing yarn sensor 110 comprises a electrically conductive helical sensor yarn (HSY). Alternatively, the stretch sensing yarn sensor 110 can comprise a composite conductive yarn (not shown). For a HSY-type stretch sensing yarn sensor 110, there is a central stretchable polyurethane core yarn 160, a helical nanofiber 162 winding around the core yarn 160, and an electrically conductive metallic coating (which can include a metal or carbon nanoparticle or nanotube or graphene) 164 on the helical nanofiber layer 162. A protective outer sheath 118 encapsulates the core yarn 160, helical nanofiber 162 and conductive metallic coating 164. The core yarn 160 can be composed of spandex or another known stretchable polyurethane material. The helical nanofiber 162 can be composed of polyacrylonitrile (PAN). The metallic coating 164 can be composed of gold, gold alloy or another suitable electrically conductive metal or metal alloy. The outer sheath 118 can be composed of polydimethylsiloxane (PDMS) or another suitable known elastomers.

The helical nanofiber 162 can be deposited onto the core yarn 160 by electrospinning a composite polymer solution including but not limited to polyacrylonitrile (PAN) dissolved in dimethylformamide (DMF). During electrospinning, the core yarn 160 is held between bias wires and rotated as the composite polymer solution is applied to the surface of the core yarn under a high DC voltage (for example as high as 1.5 kV/cm), thereby forming a helical nanofiber winding that can have for example an average diameter of 300 nm.

A thin metallic layer (e.g. few tens of nanometer of gold or other metals) of the metal coating 164 can be applied to the helical nanofiber winding 162 by plasma sputtering metal nanoparticles or nanotubes or graphene or nanowires using solution coating.

Contact electrodes (not shown) comprising Ag-coated nylon threads are electrically connected to the HSY-type stretch sensing yarn sensor 110 at desired locations over the length of the sensor yarn 160 and bound with silver paste or other known composite conductive pastes (e.g. Pelco™). The HSY-type stretch sensor 110 is then cured to reach a desired mechanical and electrical robustness, then encapsulated by the outer layer 118 by pouring and curing PDMS at a suitable temperature and time (e.g. 40° C. for 24 hrs) using a tube-shaped mold.

Experimental samples of the HSY-type stretch sensing yarn sensor 110 have been tested and demonstrated exceptional dynamic range and reliability with minimal hysteresis, being responsive to small strains as low as 0.07% and as high as 1,000%, as shown in FIG. 2(b) which illustrate a range of 150%. Additionally, the experimental samples demonstrated reliable and fast response to changes in strain and forces, as shown in FIG. 2(c); such minimal delay in operation is expected to make the yarn sensor 110 particularly suitable for fast sensing applications. Also, the experimental samples demonstrated high durability in frequent loading and unloading with reliable response after 35,000 cycles in both air and water, as shown in FIG. 2(d), which is expected to make the yarn sensor 110 washable and dryable and suitable for operation in dry and moist environments.

For a composite conductive-type stretch sensing yarn sensor 110 according to alternative embodiment, the yarn is made from a well-mixed composite paste of an elastomer with conductive nanoparticles including but not limited to carbon black, graphene, metal nanoparticles or microparticles, liquid metal or ionic liquid. Sonication and storage in cold environment may be needed to create a well-mixed solution for the paste. The paste is pressure spun or solution is wet-spun to form a continuous yarn. The diameter of the yarn is effectively determined by the diameter of the syringe and the speed of the production by the speed of the paste disposal. The paste can be disposed with a 3D printer to form complex shapes of yarns. The wet spinning in a bath enables formation of continuous and uniform yarns with given properties. Like HSY-type yarn sensors 110, contact electrode areas are formed by using soft Ag-coated threads on both sides of the sensors. The formed yarn is then encapsulated by the outer sheath 118. The deposition method can be done at high speed using robotic systems of spinning and spools to collect the yarns.

The stretch sensing yarn sensors 110 are operable to generate a desired output signal in response to stretch, deformation and pressure on the textile layers 116, 117 based on piezoresistance, piezoelectric or triboelectric principles as known in the art. The deformation can be due to the movement of a joint, interaction with an object, movement, or contraction of muscles or pulsations of a blood vessel or any other anatomical or physiological changes.

The force sensing yarn sensor 111 can be a capacitive force sensor, or a triboelectric force sensor. A capacitive force sensing yarn sensor 111 comprises two electrodes separated by an elastomer or by an elastomer perimeter and an empty separation between the two electrodes. Upon application of compressive force on certain points on the force sensing yarn sensor 111, the distance between the electrodes changes and causes a change in capacitance. Multiple force sensing yarn sensors 111 can be connected to stretchable electrodes and grouped together in rows and columns in an array. This type of force sensing yarn sensor 111 is particularly useful to detect pressure forces in different directions, including compressive, tensile and shear pressures, especially at a body part extremity such as the glover finger tips or at shoe soles. For a triboelectric force sensing yarn sensor 110, triboelectric material is be used to generate a voltage under an applied pressure. By using electro-positive and electro-negative triboelectric materials with desired size and definition in a two-layer structure that is separated by a distance, a voltage can be generated on the two electrodes connected to these layers depending on the force and separation of the two devices.

The force sensing yarn sensor 111 generates an output in response to vertical, in-plane, shear or friction forces applied when the user is interacting with an object 102 or touching surfaces or own or other users body parts. The force sensing yarn sensors 111 can be made from piezoresistive, piezoelectric or triboelectric materials and are optimized to generate stable and calibrated output in response to external forces. The timing and magnitude of the signal generated by the yarns can be used to evaluate the magnitude, location and type of force including compressive, in-plane, shear or friction.

In some embodiments, the bio-signal sensing yarn sensor 112 measures temperature and comprises electrically resistive yarns that provide changes in the resistance as a function of changes in temperature or triboelectric yarns with separation of the yarns being controlled by an elastomer that expands with temperature.

In some other embodiments, the bio-signal sensing yarn sensor 112 measures moisture (e.g. humidity or sweat) and comprises yarns made of cellulosic materials or other materials that are sensitive to absorption of water and their resistance changes with more absorption. These moisture sensing yarns can be formed by mixing elastomers with conductive materials and nanocellulose or other water sensitive materials to enable sensing of water and sweat concentration. As shown in FIG. 2(e), experimental samples of cellulosic type moisture sensors were test and demonstrated to provide better accuracy than conventional commercial humidity sensors which tend to saturate at high air water concentrations.

Alternatively, the bio-signal yarn sensor 112 can be replaced by commercially available temperature and humidity sensors (not shown), which are placed at locations in the glove that are suitable to measure body temperature and sweat when the glove is worn, e.g. at the wrist. Alternatively, a smart textile wrist band (not shown) can be provided with humidity and temperature sensors. The measurement of sweat and temperature can be used to evaluate the person's medical conditions, joint health condition, or mental condition.

In other embodiments, the bio-signal sensing yarn sensor 112 are configured to measure a bio-signal such as surface electromyography (sEMG) or electrocardiogra (ECG). Such bio-signal sensing yarn sensors 112 are composed of yarns coated with Ag directly or with AgCl using a roll-to-roll coating system for making a non-polarizing electrode yarns. Alternatively or additionally, the bio-signal sensing yarn sensors 112 are composed silver yarns coated with ionogel composite coatings (for example biocompatible chloride-based ionogel fabricated from polymerization of thioctic acid, and 1-n-Butyl-3-methylimidazolium chloride ([BMIM]Cl) ionic liquid) to provide better non-polarizing contacts to body as good as clinical gel coated solid Ag/AgCl electrodes. The yarns can be sewn, embroidered, knitted or woven to form desirable electrode sizes for electrodes or have multiple electrodes with desired spacing on the body parts. These electrodes can be used to detect electrocardiogramals, electromyography (EMG) signals, electroencephalography (EEG) signals, electrooculography (EOG) signals, or electrodermal activity (EDA) signals. The electrodes can be designed to have protrusions on the textile layers 116, 117 to have better contact with the body and use the pressure of the stretchy textile layers to have better and more stable contact with body parts such as the wrist, neck, chest, foot, or forehead. The wearable device 100 can be a wrist band, shirt, torso band, shoe insert, or headband (not shown).

The stretchy interconnects 113 can also have insulation 119 sewn between the inner 117 and outer 116 textile layers in a wavy pattern. The stretchy interconnects 113 can include a single insulated stretchy electric line or multiple insulated and/or shielded stretchy electric lines that can be twisted to form a stretchy bus line or placed beside each other in other designs. Alternatively, other integration methods for stretchable yarn sensors and stretchy interconnects can be employed such as weaving, knitting, braiding or other methods as known in the art to produce a suitably breathable and stretchable fabric with embedded interconnects and yarn sensors. The stretchable interconnects 113 are embedded in the apparel and connect sensors to the integrated circuit for data processing.

The integrated circuit 120 is integrated and encapsulated in a soft or hard embedded enclosure box 122 and can be connected to a removable integrated circuit 121 and soft or hard enclosure box 123. The embedded box 122 or removable box 123 can include a user interface 124 such as sensing or touch buttons to turn ON/OFF and/or perform other customizable functions the user desires. The embedded and removable boxes 122, 123 can each or both include secure and encrypted data storage to store some of the data collected by the yarn sensors 110, 111, 112 for wireless transfer to the data gateway and/or secured encrypted cloud or local database 103 at a later time if there is an unexpected interruption in wireless connectivity. Computer readable storage can also be provided for storing training or machine learning programs, firmware or app components locally on the integrated circuit 120 of wearable device 100. In addition, a power source is provided and protected for washability and safety of the user; for example the removable box 123 can protectively house a rechargeable battery 125 and other components that may cause hazard during washing or sanitization or not be suitable for long term storage or provide added function such as added heavier duty batteries for long term operation. The embedded enclosure box 122 can include waterproofing flanges and elastomeric bands and protective pouches and coatings for protection in frequent water use, sweat and cleaning and washing steps.

The smart textile glove 100 design can include a slit 130 that can be reversibly opened and fastened using fasteners 131 to allow better fit and easier wearing for the user. The fasteners 131 can any suitable fastener known in the art, such as magnetic fasteners as shown or reversibly sealable tape, zipper or buttons. The smart textile glove 100 can optionally include openings 140 for example at the tip of each finger or palm of hand in the textile layers 116, 117 to expose the skin of the user for better excitation of sense touch and grip for the user during handling objects that is critical for tele-rehabilitation and sport training and performance applications. The design can also include haptic actuators 141 to simulate sensor of touch for virtual or real objects by using different haptic vibrators or motors as known in the art. Another IMU sensor 114 can be embedded in the removable or embedded box 122 and 123 or integrated with a small box between the inner and outer stretchy textile.

A small area array 142 of yarn sensors 110, 111, 112 and bio-signal detectors 115 can be integrated in a part of the smart textile glove 100 that is in contact with radial artery beneath the wrist when the smart textile glove 100 is worn, to measure pressure pulse wave (PPW), heart rate, blood pressure and ECG. A custom foam or springy material (not shown) can be inserted on areas of the design to press the yarn sensor array 142 reversibly and comfortably onto the part of body when the smart textile glove 100 is worn. EMG biosignal detector yarns can also be added for adding EMG modality and improving accuracy in estimating movements and strength of the grasp. These modalities can be used in other embodiments including wrist band and arm band.

The yarn sensors 110, 111, 112, IMU sensors 114, bio-signal detectors 115 and stretchy interconnects 113 allow for integration of multi-modal sensing capability into a breathable, stretchable, comfortable, and light “smart textile” wearable device, such as the smart textile glove shown in FIG. 1(a). The yarn sensors 110, 111, 112 can each have a stretchy insulating and protective outer sheath 118 that provides washability, durability, signal isolation and can be embedded by sewing or lamination between the two stretchy inner 117 and outer 116 textile layers as shown in FIG. 1(a) to achieve desired function, stretchability, and washability. The yarn sensors 110, 111, 112 can have diameters as low as a few micrometers to a few millimeters and can be packed beside each other, woven, knitted or braided beside each other in a 2D fashion or packed, woven, knitted or braided into a 3D fashion to achieve high density of sensors with different modalities. The textile can be coated with anti-bacterial and anti-viral coatings to improve the resistance for medical and clinical applications.

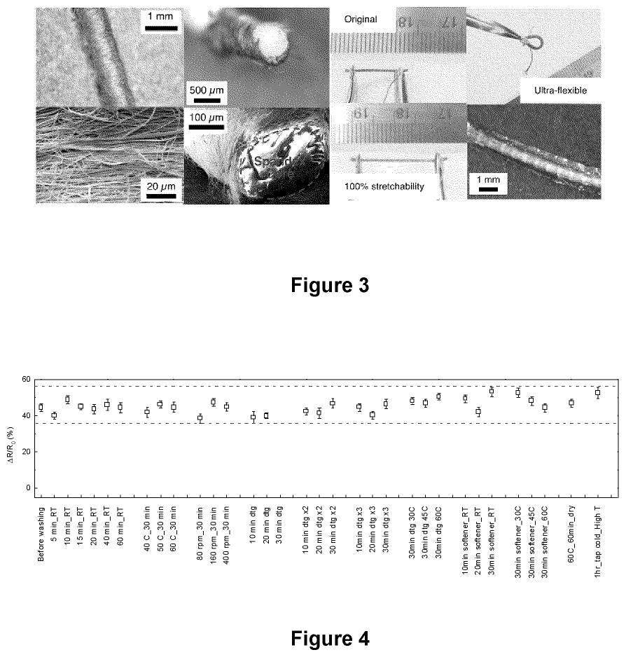

FIG. 3 depicts an example implementation of the stretch sensing yarn sensors 110, 111, 112. In this example implementation the yarn sensors are made of a stretch core spandex yarn, which is then coated by helical wrapping of metallized nanofibers. The scanning electron microscope picture showing the metallized nanofiber geometries. The yarn is then coated with a couple of elastomer coatings to provide a stretchable insulation for the stretch yarn sensors. FIG. 4 demonstrates the results of tests performed on the example sensors in an integrated apparel device undergoing different laundry washing cycles including different length of time for washing, different wash temperatures, different speed of washing, washing with detergent (dtg) for different times and temperatures, drying cycles with softener at room temperature and at different temperatures. This demonstrates that exceptional washability and durability can be achieved for the smart apparel and wearable devices as disclosed in this invention.

The multimodal sensor data obtained from the combination of the yarn sensors 110, 111, 112, IMU sensors 114 and other bio-signal detectors 115 can be used in conjunction with machine learning and Al to improve accuracy in estimation of movement parameters. For example, IMU signals are susceptible to drift with time and measurements from other modalities such as from the stretch sensing yarn sensors 110 or force sensing yarn sensors 111 or from an external camera (not shown) can be used minimize the drift and create continuous accurate movement data. This multimodality enables creating high accuracy output from both IMU and other modality sensor data. For example the stretch sensor data can be used to set specific calibration angle for the IMU sensors 114 and decrease the effect of drift in these devices.

Referring again to FIG. 1(a) and according to some embodiments, the data gateway and feedback device 103 has a processor and a non-transitory computer readable medium having stored thereon a machine learning (ML) program 200 executable by the processor to process the multimodal sensor data from the smart textile wearable device 100 to produce certain health parameter outputs for display by a software application 150 on a smartphone serving as the data gateway and feedback device 103 as shown in FIG. 1(b). Alternatively, the data gateway and feedback device 103 can be a desktop computer, an integrated computing device and display such as a tablet computer, laptop, smart watch, and virtual reality goggles, or any other general purpose computing device with a processor and programmable non-transitory memory known in the art.

Alternatively, one or both of the embedded integrated circuit 120 or removable integrated circuit 121 in the smart sensing wearable device 100 includes a processor and a non-transitory computer readable medium having stored thereon the machine learning (ML) program 200 executable by the processor, in which case the multimodal sensor data can be processed directly on the smart textile wearable device 100 and the health parameter outputs can be transmitted to the data gateway and feedback device 103 for display or notification through sound or haptic feedback on the wearable device 100 or other devices.

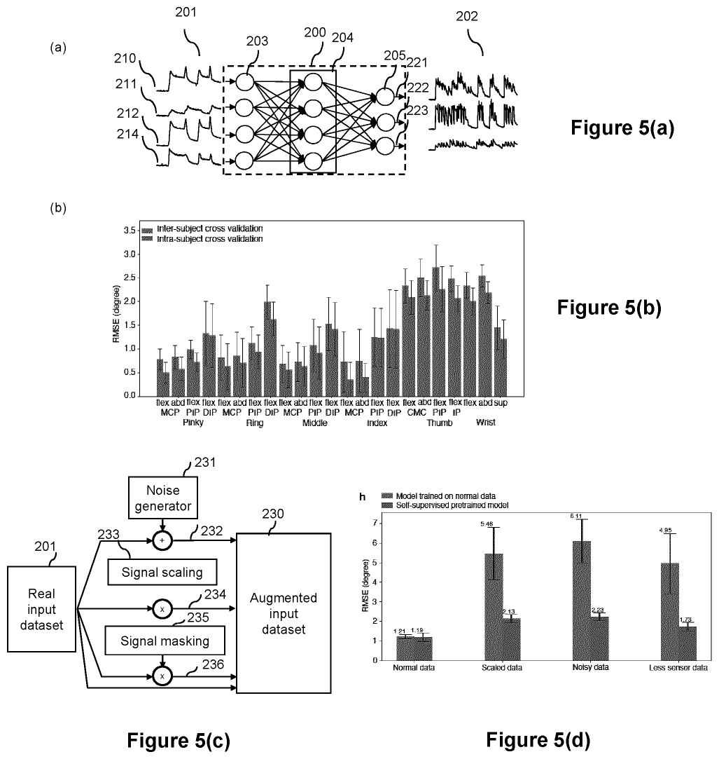

As will be described below and referring now to FIGS. 5 to 9, the core ML program 200 is pre-trained to process raw multimodal input data 201 from the sensors of the smart textile wearable device 100 in real time to produce useful health parameter outputs for display to a user such as joint angles and movements. Additionally, a pre-trained output ML program can be provided to apply certain health parameter outputs produced by the core ML program in gesture-based applications, such as using joint positions in gesture typing or in sign language recognition. This can include all static and dynamic gestures including those used in Amercian Sign Language or other Sign languages.

As shown in FIG. 5(a), raw multimodal input data 201 include: input data 210 from one or more stretch sensing yarn sensors 110, input data 211 from one or more force sensing yarn sensors 111, input data 212 from one or more bio-signal sensing yarn sensors 112, input data 214 from one or more IMUs 114, and input data from one or more other bio-signal sensing sensors such as the PPG or NIRS bio-signal detectors 115 and input data from one or more cameras. When executed, the core ML program 200 generates real-time dynamic output data 202 that contains health parameter outputs of the wearer of the smart textile wearable device, such as one or more finger joint angles 221, one or more wrist or other joint angles 222, or one or more muscle strength, object touch and force 223. The data rate can be 1, 10, 100, or 1000 Hz for IMU sensors 114, or 20 1, 10, 100, or 1000 Hz for input data for stretch sensing yarns sensors 100, or 60, 120, 240 Hz for camera frame rates, or different values as needed for different modalities of the sensors.

Different categories of input and output data can be added or removed from the core ML program 200 depending on the desired application. The core ML program 200 may include an input layer and/or a normalization layer and/or any pre-processing layer 203 for input sensors data, one or more hidden layers 204 to connect to the input layer, and one or more output layers 205 to connect to the hidden layers. FIG. 5(a) shows a schematic for these layers in generalized form of a mutli-layer perceptron. However, each layer of the core ML program 200 can include different neural networks (NN), long short-term memory (LSTM), convolutional neural network (CNN), 2-layer stacked bi-directional LSTM (Bi-LSTM), fully connected network (FC), hidden Markov model, generative neural network (GAN), or any other machine learning algorithms known to experts in the field. The core ML program 200 output is determined by real-time data as well as a sliding time window (for example 2 sec) of data to accommodate for the known trends in the time-dependent signals, which is implemented as a pre-processing in the input layer 203.

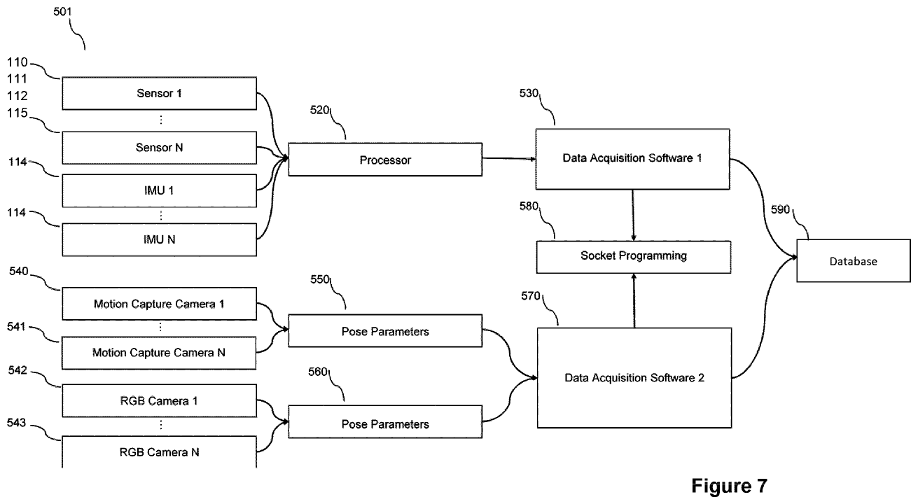

Referring to FIGS. 7-9, a training procedure of the core ML program 200 according to one embodiment involves using external devices (“ground truth devices”, not shown) to collect data relating to body metrics (“ground truth data”) while simultaneously using sensors of the smart textile wearable device 100 to collect data of the same metrics. For example, specific markers can be placed on the smart textile wearable device 100 and a high precision fixed multiple motion capture camera system can be used as a ground truth device for tracking the markers to determine hand and finger movements under different controlled scenarios. Using the motion capture camera system to track the markers on the smart textile glove 100 making random movements, performing controlled specific tasks or gestures, or interacting with objects, a training dataset is produced of certain pose parameter outputs corresponding to the tracked hand movements, such as wrist, finger and forearm joint angles and positions. The core ML program 200 can then be trained using the training dataset to estimate in real time certain dynamic pose parameter outputs 202 of a user, such as joint angles, from the multimodal sensor data collected by the smart textile glove 100 while being worn by the user. Other modalities of input can be used for training of the core ML program, including: force plate data, external force measurement systems, weight and height of the user, weight of loads used during exercises and movements, simulation results from inverse kinematic analysis of movements, input from clinical and fitness experts, input from amateur and professional coaches, assessment of clinicians, doctors, therapists, or other healthcare and sport professionals, user's opinion and experience, visual data from movement and environment of the user, visual data for other people movements, commercial body temperature measurement devices, commercial heart rate and ECG tracking, EMG electrodes, global positioning sensors, commercial moisture and sweat sensors, commercial force sensors, gym equipment settings, commercial blood pressure monitoring systems, fatigue and endurance evaluation devices and expert input, optical calibration data for LEDs and photodiodes or other validated data for other modalities.

FIG. 7 depicts an overview of a data collection procedure 501 for the core ML program training that gathers sensor data from the smart textile wearable device 100 worn by subjects in different controlled scenarios, and ground truth devices that simultaneously track markers on the smart textile wearable device 100 from the controlled scenarios to create the training dataset. To collect training data, the smart textile wearable device 100 is equipped with stretch and force sensors 110, 111, IMUs 114, and other bio-signal detectors 115. A processor 520 reads data from these sensors and transmits it to a first data acquisition software program 530.

Motion capture systems are used as ground truth devices to capture pose parameter data 550, 560 that is used for pose estimation. The motion capture systems can comprise IR-based marker tracking cameras 540, 541 and/or regular RGB cameras 542, 543 (collectively, “ground truth cameras”). The ground truth cameras 540, 541, 542, 543 capture pose parameter data 550, 560 which is transmitted to a second data acquisition software program 570, i.e. by tracking the markers on the smart textile wearable device 100. Other data can be used as ground truth or multimodal monitoring such as tagging by an expert (e.g. doctor, clinician, coach or subjective input from the user or patient) for a particular event, processed video or movement from a model to match, text input from the user, force plate sensors, weight of the loads that the user is using during an exercise, or simulation using inverse kinematics of movements for muscle forces, EMG, ECG, or other modality, global position system or other data source.

To ensure synchronization between devices, the first data acquisition software program 530 and the second data acquisition software program 570 communicate with each other using socket programming 580. Finally, both software applications transmit the collected data to a database 590. Within the database, pairs of collected data are matched with their corresponding labels to form the training dataset.

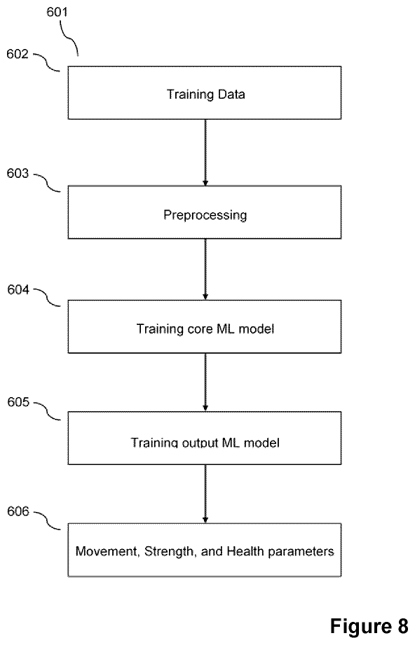

FIG. 8 illustrates the steps for training the core ML program using the collected training dataset. After collecting the training dataset 601, the dataset undergoes various preprocessing steps 603, including filtering, noise removal, normalization, and augmentation. The pre-processed dataset is then randomly divided into a training set and an evaluation set, which are used to train the core ML program 604. If further analysis is required (e.g. to use the health (pose) parameter outputs in certain gesture applications), an output ML model 605 is trained using another set of labels. Both the core ML and output ML programs 604, 605 generate intended outputs, such as postures, muscle strength, temperature, humidity, or other desired health metrics.

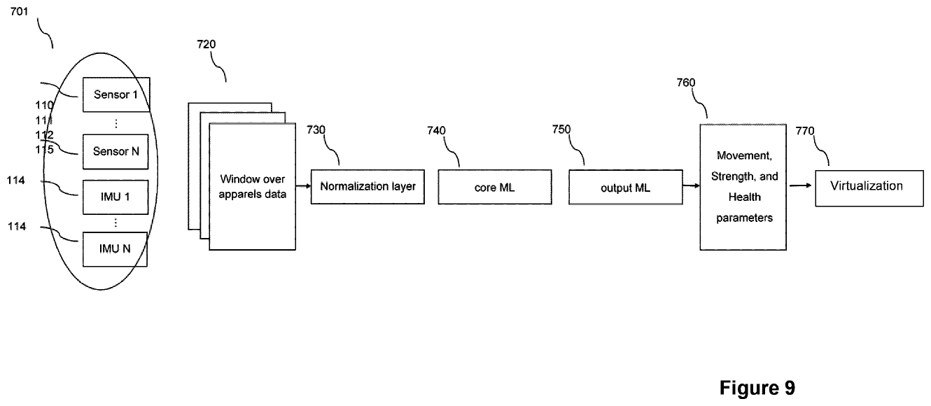

FIG. 9 presents a schematic of the real-time inference pipeline for the trained core ML and output ML programs 604, 605, utilizing data received from the smart textile wearable device 100. This proposed data flow can be implemented on hardware platforms such as FPGA or ASIC, or it can run on a processor 701. Whenever a new sensor data packet 110, 111, 112, 114, 115 is received, it is added to a buffer that holds the last N received samples 720. Subsequent processing, such as sensor value normalization or other necessary steps, is applied 730. The processed data frame is then fed into the core ML program, producing specific health parameter outputs 740. Certain health parameter outputs such as pose parameters from the core ML program 604 can subsequently be input into the output ML program 605, generating desired metrics like postures, muscle strength, temperature, humidity, or other intended metrics.

Referring to FIG. 5(b), the trained core ML program 200 can achieve a high inter-subject and intra-subject accuracy with root mean square error (RMSE) of 1.21 and 1.45, respectively, for dynamic tracking of health parameters detected by the smart textile glove 100, including: flexion/extension of finger and wrist joints, abduction/adduction for metacarpophalangeal (MCP), flexion/extension for proximal interphalangeal (PIP), distal interphalangeal (DIP) joints of pinky, ring, middle and index fingers, flexion/extension and abduction/adduction of carpometacarpal (CMC) joint, flexion/extension of proximal interphalangeal (PIP) and interphalangeal (IP) joints of thumb, and flexion/extension, abduction/adduction, supination/pronation of the wrist joint. These errors are dynamic errors of predicted angles vs those measured by a motion capture system. The trained core ML program 200 can thus provide the desired health parameter outputs even in the absence of cameras at any location and when any user wears the smart textile glove 100. The bars are average error with the standard deviations of the error for each joint.

Additionally, the core ML program 200 can be trained using augmented input data 230 as shown in FIG. 5(c) for a self-supervised pre-training algorithm. The augmented data can be (i) normal data 201 in addition to (ii) scaled data 234 for some sensor channels, generated by a signal scaling algorithm 233 or (iii) missing/masked data 236 for some sensor channels, generated by signal masking algorithm 235, or (iv) noisy data 232 for some sensor channels, generated by noise generator algorithm 231. The augmented data can also be generated using other algorithms including shuffling of some sensors, or down-sampling or up-sampling of sensor data, or any possible data augmentation techniques for multi-channel time series data. This pre-training algorithm can enhance the robustness of the core ML program 200 to changes in sensor behaviour caused by malfunction of some sensors, operational variation such as scaling or noise due to changes in environmental conditions such as temperature, sweat, fit to use body in a session or between sessions. As shown in FIG. 5(d), the self-supervised pre-training model can significantly enhance the accuracy of the core ML program in the presence of external sources of noise.

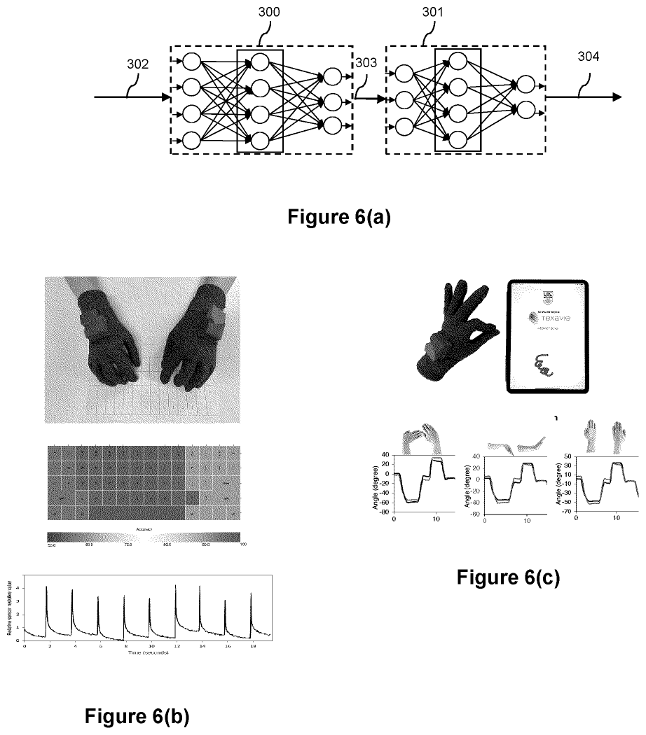

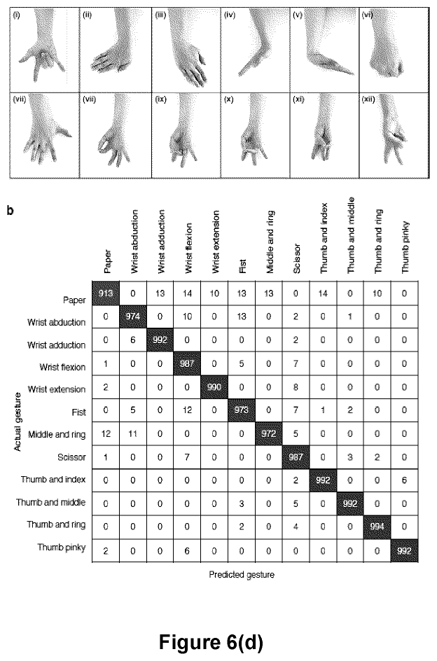

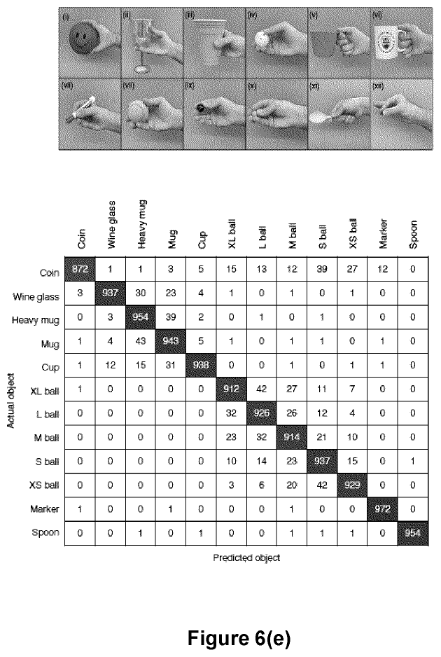

Another embodiment is illustrated in FIG. 6(a)-(e), showing that the core ML program 300, like what was described in FIG. 5(a)-(d), can be connected to a new output ML program 301 to train for specific health parameter outputs 303 used in certain gesture based applications, while keeping the core ML program 300 as before functioning on the real-time data from the sensors 302 of the smart textile wearable device 100. The output ML program 301 gets input from both the output 303 of the core ML program 300 as well as data from a subset or all of the sensors 302 or from other sensors as needed. The output ML program 301 can have the desired input, hidden and output machine learning layers as described for the core ML program 300 previously. For example, the output ML program 301 can be specifically trained for using sensor inputs in certain gesture based applications, such as predicting keys during typing on a mock paper keyboard (FIG. 6(b)) or on any surface. This keyboard can be in any shape or form on a flat or curved surface. Here as an example, a mock keyboard that is printed on a piece of paper is placed on a desk in front of a user wearing a pair of the smart textile gloves 100. The user types on the mock keyboard and the desired output 304 includes the keys that are pressed and its timing are sent to a software application. In this case, the core ML program 300 generated from previous trainings is used as before to predict the angle of all joints of the fingers in the smart textile gloves 100 (pose parameter outputs), and the output ML program 301 is trained to assign these joint angles to specific pressing of keys based on the pose of the hand as well as sensing clicks by the sensors at the tip of the fingers (see FIG. 6(b)). The resulting performance can be highly accurate typing with color coded key detection accuracy as shown in FIG. 6(b). In this case, the core ML program 300 is not trained anymore, but the output ML program 301 is trained for typing for a specific user. In this embodiment, input from a camera placed on AR/VR goggle or user computer, phone or tablet can be used to provide calibration for the area or adjusting the area of the keyboard as well as asking user to press a few keys for calibration before starting to type. Another example of a gesture-based application is to train the output ML program 301 to respond to hand drawing in air based on the wrist angle and pinching detection for thumb and different fingers to select different colors (FIG. 6(c)). In this case, the desired application outputs include angles of the wrist (abduction/adduction, flexion/extension, and supination/pronation) and detection of pinching using force and stretch sensors (similar to FIG. 6(b)). Another example can include accurate complex gesture recognition (FIG. 6(d)), where the output ML program 301 is trained to recognize complex gestures with a high accuracy as shown in the confusion matrix for 12 complex gestures in FIG. 6(d). Another example application can be as shown in FIG. 6(e) is to recognize specific objects from the hand shape and grasp of the user holding the object with a high accuracy shown by the illustrated confusion matrix. In this case the output ML program 301 is trained to recognize 12 objects rapidly without retraining of the core ML program 300. The output model can be trained to provide output on the amount of force each finger or palm of hand is applying during holding or grasping of an object.

Additionally, the output ML program can be trained for specific health parameter outputs to provide feedback in real-time or long term. For example, a software application (App) 150 executed on a user device can be used for real-time feedback as shown in FIG. 1(b). The App 150 can help organize a personalized exercise plan for the hands and visualize progress for an individual session 151 or over the length of few sessions or over weeks or months of exercise therapy 158 and 159. The App 150 can provide an avatar 152 that helps guide the user through exercises, for which the movements and speed can be adjusted based on the real-time data from the smart glove 100. The avatar 152 can be used to guide the timing and sequence of an exercise plan for which the detail can be presented and shown to the user for example using 151. The App 150 can be used to focus the user's attention on specific timing or focus points of an individual exercise as shown by 153, which can be actively updated from real-time data of the smart textile glove 100. The App 150 can provide accurate target points 154 for the user to achieve during a specific exercise, which in this case for an example embodiment is set as level of grasping force and angle as charted by clinical guidance during a grasping of a tennis ball exercise. This can be any motion, pose or timing conditions as measured accurately by the smart glove or other embodiments of this invention. The App 150 can provide information about symmetry of movement 155 by showing accurate ROM or finger angles for both left and right hands during a specific dynamic exercise and provide feedback to user to improve their symmetry by modifying their pose, form of movement, diagnosis of user asymmetric tendencies or muscles or training muscles to obtain better symmetry. The App 150 can be used to select individual muscles of fingers or wrist 156 for real-time feedback in terms of strength, force, volume that are dynamically monitored during an exercise. Detailed information about peak force, torque, strength and symmetry of muscles for left and right hands can be shown 157. Other parameters such as intensity of exercise, strengthening of individual finger muscles over time, exercise effectiveness, improvements in forms, suggestion of other specific exercise or routines, pinching, writing, throwing, pouring, typing, pulling, exercise smoothness or other useful information for evaluation of exercise or daily activities can be displayed in real-time or set as personalized targets for the user. The feedback to the user can be set as progression of strength or other parameters for users over several sessions during weeks or months of therapy. The exercise plan and therapy targets can be set by an expert, including but not limited to a physiotherapist, a specialist, a clinical expert, an occupational therapist, a sport medicine specialist, a sport injury therapist, a fitness coach, or anyone with expertise for the conditions of a patient. The analyzed data can be presented to the expert in real-time in clinic working with the patient or remotely as the expert is actively supervising a therapy session or after one or few unsupervised remote therapy sessions. The App 150 can also help in visualizing the movements in different scenarios or games or interactive games that help motivate the user in achieving therapy goals or feeling more comfortable.

The data generated during exercise sessions by a user using the smart textile wearable device 100 and the App 150 can be securely encrypted and stored locally on the smart textile wearable device 100 or a local server or a cloud server, a blockchain, or any server available for movement and health data or electronic medical records (EMR). The core ML program 300 or output ML program 301 can be trained securely using federated learning techniques to enable collaborative learning from different users to improve accuracy of algorithms for all users without risk for privacy or security of the user. In addition, the federated learning can help in overcome data heterogeneity challenges for different users in creating fair and robust core and output ML programs 300, 301. The core and output ML programs 300, 301 trained using federated learning can be stored in secure and encrypted locally on the smart textile wearable device 100 or a local server or a cloud server, a blockchain, or any server available. Local modifications and user dependent models can be implemented locally to tune the output of the algorithms for specific user.

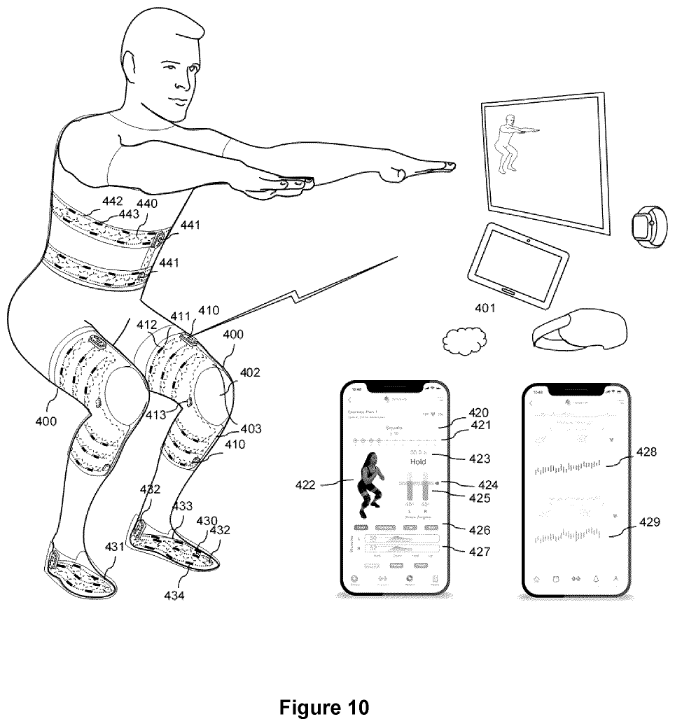

FIG. 10 illustrates other embodiments of the smart textile wearable device 100, including smart textile knee sleeves 400, smart textile shoe insoles 430, 431 and smart torso band(s) 440. The smart knee sleeves 400 are embedded with different modalities of yarn sensors including stretch sensing yarn sensors 410, force sensing yarn sensors 411, temperature sensing yarn sensors 412, and sweat sensing yarn sensors 413, which cover the surface of the thighs and calves and IMUs 414 that are embedded on the top and bottom part of the sleeve. The smart knee sleeves 400 can have an opening 402 for easier wearing and comfort, be pulled in view of its stretchability or can be reversibly opened at slit 410 and then fastened and tightened to a comfortable tightness using bands or other fastening mechanisms. The knee sleeves 400 can wirelessly connect to the data gateway and feedback system 401 in a similar manner to the smart textile gloves 100. The smart yarn sensors can be embedded between an inner and an outer textile layers of the knee sleeves or knitted, woven, or braided in stretchy textile. The smart knee sleeves 400 can have surface modifications such as elastomer coatings on the inner textile to improve friction to the thigh or other parts of the legs to improve stability and avoid slippage during different movements and intense exercises.

The smart knee sleeves 400 are worn by the user and used during different exercise or movements for real-time or long-term feedback from the data gateway and feedback system 401. A core ML program, similar to what was described for previous embodiments, is trained to generate output related to the different desired parameters including knee joint angle for both legs, or angles not covered by the smart knee sleeves for example hip or ankle angle including accurate Range of Motion of left or right knees or other lower body joints, valus/vargus movements, or individual muscle volume and force for hamstrings, quads, calves and shin muscles. The core ML program can use data from both IMUs 414 and stretch and force sensing yarn sensors 410, 411 to estimate angles and poses accurately. The multimodal sensing functionality help in addressing technical challenges such as drift in IMUs or sensors. The model is trained based on a gold-standard dataset gathered using accurate motion capture cameras, forces sensors, force plates and in the presence of different exercise equipment to train different muscles in a similar manner as described for other embodiments.

After the training of the core ML program, an output ML program, like what was described for previous embodiments, can be trained for specific health parameter outputs to provide feedback in real-time or long term. For example, a software application (App) 420 can be used for real-time feedback as shown in FIG. 5(b). The App 420 can help organize a personalized exercise plan and visualize progress for an individual session 421 or over the length of few sessions or weeks or months of exercise therapy 428 and 429. The App 420 can provide an avatar 422 that helps guide the user through exercise, for which the movements and speed can be adjusted based on the real-time data from the knee sleeves 400. The avatar can be used to guide the timing and sequence of an exercise plan for which the detail can be presented and shown to the user for example using 421. The App 420 can be used to focus user on specific timing or focus points of an individual exercise as shown by 423, which can be actively updated from real-time data of knee sleeves. The App 420 can provide accurate target points 424 for the user to achieve during a specific exercise, which in this case for an example embodiment is set as time and angle of knee joints during a squat exercise. This can be any motion, pose or timing (including timing of different stages of exercises) conditions as measured accurately by the knee sleeves or other embodiments of this invention. The App 420 can provide information about symmetry of movement 425 by showing accurate ROM or knee angles for both left and right legs during a specific dynamic exercise and provide feedback to user to improve their symmetry by modifying their pose, form of movement, diagnosis of user asymmetric tendencies or muscles or training muscles to obtain better symmetry. The App 420 can be used to select individual muscles or muscle groups 426 for real-time feedback in terms of strength, force, volume that are dynamically monitored during an exercise. Detailed information about peak force, symmetry of muscles for left and right legs can be shown 427. Other parameters such as intensity of workout, repetitions of specific moves or exercises, timing of different stages of exercises including rest, loading movement, holding and unloading movement, consistency in timing of different stages of exercises, speed of movement of joints, anlges at which maximum force and strength are applied, time at maximum force, isometric and isotonic movement times and magnitudes of contractions, exertion levels, repetition, exertion and endurance levels, strengthening of individual muscle groups over time, exercise effectiveness, improvements in forms, suggestion of other specific exercise or routines, walking, running and gait parameters, output power, calories, exercise smoothness or other useful information for evaluation of exercise or daily activities can be displayed in real-time or set as personalized targets for the user. The feedback to user can be set as progression of strength or other parameters for users over several sessions during weeks or months of therapy. The exercise plan and therapy targets can be set by an expert, including but not limited to a physiotherapist, a specialist, a clinical expert, an occupational therapist, a sport medicine specialist, a sport injury therapist, a fitness coach, or anyone with expertise for the conditions of a patient. The analyzed data can be presented to the expert in real-time in clinic with patient or remotely as the expert is actively supervising a therapy session or after one or few unsupervised remote therapy sessions. The App can also help in visualizing scenarios that help motivating the user in achieving therapy goals or feeling more comfortable.

The output and core ML programs can be fed with other data from other embodiments such as smart textile shoe insoles 430 and 431, described below, or smart textile gloves 100 or any other wearable device that provides data about physiological and health parameters. Such health data enables dynamic evaluation of the progress of the user for achieving cardio rehabilitation or other performance goals.

The smart textile shoe insoles 430 and 431 shown in FIG. 10 are another example embodiment of the smart textile wearable device, where different modalities of sensors are used including IMU sensors 432 placed for example at the hill and the toes of each insole, pressure sensing yarn sensor 433, stretch sensing yarn sensors, bio signal sensing yarn sensors 434 for sensing sweat and temperature of the user's feet during walking, working, running, exercise with weight, skiing, or other activities. The core ML program can be trained to output general movement parameters such as pace, gait, landing pressure and other parameters. The output ML program can be trained to provide specific output such as symmetry in training, real-time training feedback or long-term strengthening performance. The parameters of interest can be communicated to the user using the App 422 in real-time or after many sessions of use and evaluation.

The parameters include the strength of muscles, symmetry of muscle group strengths, walking pace, gait parameters, cadence, abnormal positioning of feet (inward, outward), and the fit and suitability of shoes for the user.

The smart torso band or bands 440 shown in FIG. 10 are another example embodiment of the smart textile wearable device, where different modalities of sensors are used including IMU sensors 441 located at different locations at the front, side or back, stretch sensing yarn sensors 442, and bio signal yarn sensors 443 for sensing force, sweat, and temperature. The core ML program can be trained to provide output for spine angle during exercises in different directions, movement of core muscles, shape and form of waist and belly, breathing rate, heart rate, heart rate variability, ECG, fetus movement for pregnant mothers, or other desired parameters. The output ML program can be trained to provide objective feedback for example for exercise modification, health of the individual, intensity of exercise or health of the fetus or unborn child.

Other embodiments of the smart textile wearable device include smart textile elbow sleeves, wrist bands, shirts, tights, socks, shoulder sleeves or neck sleeves or hats (not shown). Additionally, the smart textile wearable device can be embodied as other apparel, such as a smart textile shirt, pants or socks or body suites. In these embodiments, the core ML program can be trained to output variety of overall body movement and joint angles as well as muscle strength and symmetry. The output ML program can be used to provide feedback on specific parameters during exercises and movement of the user during certain applications. Notably, the overall form of smart textile apparel can be configured for ease of use of the wearable device, e.g. fast wearing and set up of the device without complexity.

The data generated during exercise sessions by a user using the smart knee sleeves 400, smart shoe insoles 430 and 431, and chest and torso bands 440 and any other form of smart textile apparel and the App 420 can be securely encrypted and stored locally on the device or a local server or a cloud server, a blockchain, or any server available for movement and health data or electronic medical records (EMR). The core ML program 300 or output ML program 301 can be trained securely using federated learning techniques to enable collaborative learning from different users to improve accuracy of algorithms for all users without risk for privacy or security of the user. In addition, the federated learning can help in overcome data heterogeneity challenges for different users in creating fair and robust core and output ML programs. The core and output ML programs trained using federated learning can be stored in secure and encrypted locally on the device or a local server or a cloud server, a blockchain, or any server available. Local modifications and user dependent models can be implemented locally to tune the output of the algorithms for specific user.

The word “a” or “an” when used in conjunction with the term “comprising” or “including” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

The terms “coupled”, “coupling” or “connected” as used herein can have several different meanings depending on the context in which these terms are used. For example, as used herein, the terms coupled, coupling, or connected can indicate that two elements or devices are directly connected to one another or connected to one another through one or more intermediate elements or devices via a mechanical element depending on the particular context. The term “and/or” herein when used in association with a list of items means any one or more of the items comprising that list.

As used herein, a reference to “about” or “approximately” a number or to being “substantially”equal to a number means being within +/−10% of that number.

While the disclosure has been described in connection with specific embodiments, it is to be understood that the disclosure is not limited to these embodiments, and that alterations, modifications, and variations of these embodiments may be carried out by the skilled person without departing from the scope of the disclosure.

It is furthermore contemplated that any part of any aspect or embodiment discussed in this specification can be implemented or combined with any part of any other aspect or embodiment discussed in this specification.

1. A wearable device for tracking one or more body metrics, comprising:
a textile layer configured to be worn on a body part of a user;
at least one body metric sensor including a yarn sensor attached to a location on the textile layer suitable to track a target body metric when the textile layer is worn by the user, and
an integrated circuit with at least one conductive stretchable interconnect communicatively coupled to the at least one body metric sensor.

2. The wearable device as claimed in claim 1 wherein the yarn sensor comprises one or more of a stretch sensing yarn sensor, a force sensing yarn sensor, a temperature sensing yarn sensor, a moisture sensing yarn sensor, a surface electromyography (sEMG) bio-signal yarn sensor or an electrocardiograma-signal yarn sensor.

3. The wearable device as claimed in claim 2 wherein the stretch sensing yarn sensor comprises an electrically conductive helical sensor yarn comprising a central stretchable core yarn, a helical nanofiber winding around the core yarn, a metal coating on the helical nanofiber winding and core yarn, and an outer sheath encapsulating the core yarn, helical nanofiber winding, and metal coating.

4. The wearable device as claimed in claim 3 wherein the core yarn has a composition comprising polyurethane, the helical nanofiber winding has a composition comprising polyacrylonitrile (PAN), the metal coating has a composition comprising gold or an alloy thereof, and the sheath has a composition comprising an elastomer.

5. The wearable device as claimed in claim 2 wherein the stretch sensing yarn sensor comprises a composite yarn having a composition comprising conductive nanoparticles and an elastomer.

6. The wearable device as claimed in claim 2 wherein the force sensing yarn sensor is a capacitive force sensor comprising a pair of electrodes separated by an elastomer or by an elastomer perimeter with an empty separation between the electrodes, or a triboelectric force sensor.

7. (canceled)

8. The wearable device as claimed in claim 2 further comprising multiple force sensing yarn sensors grouped together in an array that is attached to a location on the textile layer in the vicinity of a body part extremity when worn by the user.

9. The wearable device as claimed in claim 2, wherein the temperature sensing yarn sensor comprises electrically resistive yarns that changes resistance as a function of temperature change, or triboelectric yarns connected to an elastomer that changes dimension as a function of temperature change, and wherein the moisture sensing yarn sensor has a composition that changes resistance as a function of water absorption.

10. (canceled)

11. The wearable device as claimed in claim 1 further comprising at least one inertial measurement unit (IMU) sensor attached to the textile layer and operable to output spatial data of the body part wearing the wearable device.

12. The wearable device as claimed in claim 1 wherein the at least one body metric sensor further comprises a photoplethysmography (PPG) or near-infra-red sensor (NIRS) bio-signal detector attached to the textile layer in the vicinity of a blood vessel when worn by the user, and/or a bio-signal sensing yarn sensor comprising Ag or AgCl coated yarns, and operable to measure one or more of electrocardiography (ECG) signals, electromyography (EMG) signals, electroencephalography (EEG) signals, electrooculography (EOG) signals, or electrodermal activity (EDA) signals.

13. (canceled)

14. The wearable device as claimed in claim 1 further comprising multiple yarn sensors placed arranged in an array and attached to a location on the textile layer in the vicinity of a body part extremity when worn by the user.

15. The wearable device claimed in claim 1 wherein the integrated circuit comprises a wireless transmitter.

16. The wearable device as claimed in claim 1, wherein the textile layer is configured to be one or more of a glove, wrist band, knee sleeve, shirt, tights, socks, shoe insole, or torso band.

17. A system for tracking, processing and displaying body metrics, comprising;
a wearable device as claimed in claim 2; and
a computing device comprising a processor and a non-transitory computer readable medium having stored thereon a trained core machine learning (ML) program executable by the processor to receive raw body metric data from the at least one body metric sensor, correlate the raw body metric data with a corresponding health parameter output stored in a first training dataset, and display the corresponding health parameter output.

18. The system as claimed in claim 17, wherein the computing device further comprises an output ML program communicative with the core ML program and executable by the processor to receive health parameter output from the core ML program, correlate the health parameter output with a corresponding gesture in a second training dataset, and display the corresponding gesture in a gesture-based application.

19. The system as claimed in claim 17 wherein the textile layer is configured as a knee sleeve, the one or more yarn sensors include a stretch sensing yarn sensor, a force sensing yarn sensor, and an IMU sensor, and the health parameter output includes one or more of knee joint angle, range of motion of knee or other lower body joints, valus/vargus movements, speed and torque of movements, timing of movements, repetition of movements, consistency in timing, or individual muscle volume, force, strength, activity, and symmetry between two legs.

20. The system as claimed in claim 17, wherein the textile layer is configured as a shoe insert, the one or more yarn sensors include a stretch sensing yarn sensor, a force sensing yarn sensor, and an IMU sensor, and the health parameter output includes movement parameters including steps, power, pace, gait, and landing pressure and symmetry between two feet, or
wherein the textile layer is configured as a torso band and the one or more yarn sensors include a stretch sensing yarn sensor, force sensing yarn sensor, temperature sensing yarn sensor, moisture sensing yarn sensor, sEMG bio-signal yarn sensor and ECG bio-signal yarn sensor, and the health parameter output includes spine angle, movement of core muscles, shape and form of waist and belly, breathing rate, heart rate, heart rate variability, blood pressure and ECG.

21. (canceled)

22. The system as claimed in claim 17 wherein the textile layer is configured as a glove, and the one or more yarn sensors include a stretch sensing yarn sensor, force sensing yarn sensor, and the health parameter output includes wrist angle, finger joint angles, finger pinching force, grasp force, and object touch force.

23. The system as claimed in claim 17, wherein the textile layer is configured as a wrist band comprising a photoplethysmography (PPG) or near-infra-red sensor (NIRS) bio-signal detector and a stretch sensing yarn sensor, force sensing yarn sensor, sEMG bio-signal yarn sensor and ECG bio-signal yarn sensor, and the health parameter output includes blood pressure, heart rate, hands or fingers movement or force, and ECG.

24. The system as claimed in claim 17, wherein the first training dataset is produced by simultaneously collecting body metric data tracked by an external device and by the at least one body metric sensor on the wearable device, while the wearable device is performing specified movements with known health parameter outputs.

25. The system as claimed in claim 24, wherein the external device comprises one or more motion capture cameras and the wearable device comprises markers, and wherein during training of the core ML program, the one or more motion camera cameras track the markers when the wearable device is performing the specified movements.

26. The system as claimed in claim 25 wherein the wearable device comprises at least one IMU sensor and a stretch sensing yarn sensor or a force sensing yarn sensor or a camera and the core ML program is further executable by the processor to calibrate the IMU using sensor data collected from one or more of the stretch sensing yarn sensor, force sensing yarn sensor or external camera, by comparing the collected sensor data to IMU signals to determine IMU signal drift.

27. The system as claimed in claim 17 wherein the wearable device further comprising a liquid sealed embedded enclosure attached to the textile layer and enclosing the integrated circuit and/or a non-transitory computer-readable storage.

28. The system as claimed in claim 27 wherein the computing device is remote from the wearable device and the integrated circuit comprises a wireless transmitter for wirelessly communicating with the computing device.

29. The wearable device as claimed in claim 27 further comprising a removable enclosure removably attached to the wearable device and enclosing a power source and/or a non-transitory computer-readable storage.

30. (canceled)
